(19) United States
(12) Patent Application Publication
(10) Pub. No.: US 2005/0148673 A1
(43) Pub. Date: Jul. 7, 2005

(54) Prolonged administration of NMDA antagonist and safener drug to alter neuropathic pain condition

(76) Inventors: Ronald E. Harbut, Page, AZ (US); Graeme E. Correll, Mackay (AU); John W. Olney, Ladue, MO (US)

Correspondence Address: Patrick D. Kelly, # 403, 11939 Manchester, St. Louis, MO, 63131, US

(21) Appl. No.: 10/373,433

(22) Filed: Feb. 23, 2003

Related U.S. Application Data
(60) Provisional application No. 60/395,448, filed on Jul. 11, 2002.

(51) Int. Cl. A61K 31/137

(52) U.S. Cl. 514/650

(57) ABSTRACT

A drug that inhibits NMDA receptors (such as ketamine, a surgical anesthetic) is continuously administered to patients suffering from neuropathic pain. Unless the NMDA antagonist drug has inherent safening activity, this treatment requires a “safener” drug to prevent the neurotoxic side effects of NMDA antagonists. One class of safener drugs that increase the efficacy of the treatment include alpha-2 adrenergic agonists, such as clonidine. The treatment lasts for several days and nights, continuously. A maximum tolerated dosage is titered for each patient, such as by observing slurring of speech, and the patient does not lose consciousness except during normal sleep. Magnesium and/or drugs that inhibit ketamine-degrading enzymes can also be used. Patients who suffered for years from chronic intractable pain emerged from this treatment with apparently permanent relief, or with lasting reductions in their levels of pain.

### PRIORITY CLAIM

This invention claims the benefit, under USC 119(e), of provisional patent application 60/395,448, filed on Jul. 11, 2002.

### FIELD OF THE INVENTION

This invention is in the field of pharmacology and pain management, and more particularly relates to the use of a drug such as ketamine, which normally is used as a short-acting surgical anesthetic, in a different type of treatment to provide lasting relief from neuropathic pain.

### BACKGROUND OF THE INVENTION

Ketamine is the common name for a drug that is widely used as a surgical anesthetic. Two of the more important traits of ketamine are as follows:
(i) it is cleared from the bloodstream relatively rapidly, and therefore it enables anesthesiologists to bring a sedated and unconscious patient out of anesthesia more rapidly than can be achieved by using many other types of anesthetics that have a longer duration of action; and,
(ii) it is a fairly potent NMDA antagonist drug (i.e., it exerts its effects by suppressing and reducing activity at the so-called NMDA subclass of a larger class of neuronal receptors known as glutamate receptors). The term “NMDA antagonist” is used interchangeably herein with terms such as “NMDA receptor blocker” or “NMDA blocker drug”.

Because of its activity in blocking NMDA receptors, ketamine has valuable therapeutic potential, but it also poses a significant risk of damaging certain specialized classes of neurons in a patient's brain. This risk of brain damage arises because of certain traits of glutamate receptors, including NMDA receptors.

“Glutamate” refers to the ionized form of glutamic acid, an important amino acid. The ionized form and the “free acid” form of this amino acid are both present, in equilibrium, in any aqueous fluid. In fluids that are present in cells and blood, which are not highly acidic, the ionized form (glutamate) is present at much higher concentrations than the acid form (glutamic acid). However, they are simply two versions of the exact same compound, and they coexist, in equilibrium, in all cells and cellular fluids.

In all vertebrate animals, glutamate has two entirely different functions. First, it is one of the twenty “primary” amino acids that are used as the building blocks to create all protein, in all living things on earth. As such, glutamate is ubiquitous in nature, and is found in all living cells.

However, the role of glutamate as an amino acid, for building proteins, is not its only crucially important biological role. In vertebrates, it also is an excitatory neurotransmitter. This means that glutamate molecules are used by neurons to transmit nerve impulses from one neuron to another, via a synapse (synaptic junction) between a transmitting neuron and a receiving neuron. Glutamate and acetylcholine are the two most important excitatory neurotransmitters, and glutamate receptors are crucially important, throughout any mammalian central nervous system.

It should be noted that glutamate receptors can also be activated by another primary amino acid, called aspartic acid (its ion is called aspartate). Therefore, glutamate receptors are sometimes referred to as “excitatory amino acid” (EAA) receptors. However, aspartate is used only rarely as a neurotransmitter, while glutamate is a major CNS neurotransmitter. Therefore, EAA receptors are referred to more commonly as glutamate receptors.

There are two major families of glutamate receptors, called ionotropic and metabotropic receptors. The NMDA receptor subclass belongs to the ionotropic family, which was given that name because these receptors control ion channels, which allow certain types of ions to pass through the membranes that enclose neurons.

There are three subclasses of ionotropic glutamate receptors, and each subclass is named after a certain type of “probe drug” which selectively activates that particular subclass. NMDA receptors were named after a compound called N-methyl-D-aspartate (NMDA), because scientists learned in the 1980's that NMDA will strongly activate that one subclass of glutamate receptors, without having any substantial activity at the other two subclasses of glutamate ionotropic receptors. The NMDA compound normally is not present inside mammalian brains, and it is not used as an actual drug for treating any medical conditions, since it can cause severe convulsions, due to its very potent activity in triggering NMDA receptors. However, since NMDA was the first known compound that could be used to clearly identify and distinguish one particular subclass of glutamate receptors, those receptors were designated as NMDA receptors.

The other two subclasses of ionotropic glutamate receptors are called kainate receptors, and AMPA receptors, because of their reactions with two other probe drugs. They are much less widespread than NMDA receptors, and they are much more similar to each other than to NMDA receptors (i.e., both kainate and AMPA receptors can be triggered by a number of probe drugs that do not trigger NMDA receptors; this type of overlapping receptor activity is often referred to as “cross-affinity”). Therefore, kainate and AMPA receptors are often referred to collectively as “non-NMDA” receptors.

AMPA receptors formerly were called quisqualate (or QUIS) receptors, since they were first discovered to be activated by a probe drug called quisqualic acid. However, quisqualic acid also triggers a completely different class of metabotropic receptors. Therefore, to avoid confusion, researchers today use a more selective probe drug, called alpha-amino-3-hydroxy-5-methylisoxazole-4-propionic acid (abbreviated as AMPA), as the standard probe drug for evaluating activity at AMPA receptors.

Although glutamate plays an absolutely crucial and highly beneficial role as the predominant excitatory neurotransmitter inside the brain, its activity as a neurotransmitter also has a dangerous side, which can be explained as follows.

Under normal and healthy conditions, when a neuron is transmitting a nerve signal to another neuron, a molecule of glutamate is released by the transmitting neuron, at a synaptic junction between the two neurons. The glutamate molecule enters the fluid that fills the synaptic junction between the two neurons, and the glutamate briefly binds to the exposed portion of a receptor protein that is embedded in the cell membrane on the surface of the signal-receiving neuron. This binding reaction (between the glutamate and the receptor protein) leads to the opening of ion channels through the membrane that encloses the signal-receiving cell. This opening of ion channels in the membrane allows sodium (Na⁺) and calcium (Ca⁺⁺) ions to flow into the neuron. The glutamate molecule is then released from the receptor protein, and it floats back into the fluid that fills the synaptic junction between the two neurons.

When a free glutamate molecule is released by a synaptic receptor protein, it is quickly grabbed by a transporter protein, as part of a series of reactions that cause the glutamate molecule to be pumped back into the interior of the neuron that originally released that glutamate molecule. This allows the signal-transmitting neuron to recycle and reuse that same glutamate molecule, in a later “firing” event. This entire four-step process (i.e., glutamate release by a signal-transmitting neuron; binding of the glutamate to a receptor on the signal-receiving neuron; release of the glutamate by the receptor; and transport of the glutamate back into the signal-transmitting neuron) occurs within milliseconds. Although it is usually highly efficient, it is not perfect, and free glutamate molecules occasionally escape from synaptic junctions. Then they do, they are usually absorbed by support cells called “glial cells”, which are present in brain and spinal tissue, but which cannot send or receive nerve signals.

Ketamine functions as a surgical anesthetic by blocking the activity of glutamate molecules at NMDA receptors. Because NMDA receptors are so common and widespread throughout the CNS of any human or other mammal, even a partial blockade of the NMDA receptor system, by a drug such as ketamine, can render a patient completely unconscious, during surgery.

In addition to that type of recognized medical use as a surgical anesthetic, it has been recognized for years that NMDA receptor blocker drugs also have a second potentially large and important medical use, because they may be able to help reduce and minimize brain damage after a stroke, cardiac arrest, near-suffocation, head or spinal injury, or other crisis that causes an interruption of blood flow or oxygen supply to the brain. Although this potential use for NMDA blocker drugs (which includes ketamine) is not directly related to the pain-control use disclosed herein, the reader should be familiar with that field of research involving NMDA receptor blockers, to better understand this current invention.

To understand the potential use of NMDA blockers to reduce brain damage following a stroke or other crisis, one must focus on the transporter system that, in a healthy brain, pumps free glutamate molecules back into the interiors of the neurons that released those glutamate molecules. That transporter system requires energy, to drive the system and enable those pumping reactions to keep moving forward. If the energy supply in a certain part of the brain or spine is disrupted, by a crisis such as a stroke, cardiac arrest, or other trauma that cuts off the blood or oxygen supply to that region of brain or spinal tissue, then the glutamate transporter system will run out of energy, and the pumping reactions will stop.

If that happens, excess glutamate molecules will begin to accumulate in the fluids that fill the synaptic junctions between neurons. These free glutamate molecules will quickly begin to make the crisis even worse, because they will begin reacting again and again, in an uncontrolled and dangerously excessive manner, with the same glutamate receptors (including NMDA receptors) they had been binding to (as useful and essential neurotransmitters), before the crisis began. This will severely over-stimulate the receptor-bearing neurons, and will drive them rapidly to a point of exhaustion, because each time a neuron is triggered to fire, in a region of brain or spinal tissue that is suffering a crisis, it uses up even more of the rapidly-dwindling energy supply.

This process will quickly begin to cause nerve cells to literally begin dying from exhaustion. The “resting” state for any neuron is actually a high-energy state, in which the neuron is perched on a high-energy plateau, where it is (in effect) fully loaded, primed, and ready to fire off another nerve signal. A neuron reaches this ready-to-fire status by using energy to pump ions into and out of the neuron. This mainly involves pumping sodium ions, Na⁺, out of the neuron, to create a negative charge inside the cell; however, potassium, chloride, and calcium ions also play important roles in this process. The ion pumping process continues until the neuron establishes a voltage gradient of roughly −90 millivolts (mV) across its outer membrane (different types of neurons establish voltages that range from about −70 to about −100 mV, when at rest). This voltage gradient across a neuron's outer membrane drops substantially, during each “firing” or “depolarization” event. As soon as that happens, the neuron will begin using its energy supply to begin the ion pumping process again, as it tries to regain its high-voltage resting state as quickly as possible so that it will be ready for the next nerve signal.

Brain and spinal tissue do not and cannot keep any spare or reserve supplies of energy, or oxygen. Therefore, excessive levels of over-excitation by glutamate molecules that begin accumulating in synaptic junctions, in a crisis such as a stroke or cardiac arrest, will cause the affected neurons to become severely and even lethally stressed, very rapidly. If the stress continues for more than a few minutes, it will begin to literally kill the exhausted and depleted neurons. This type of injury is called “excitotoxic” damage, and it has been shown that accumulation of excess glutamate, at NMDA receptors, is the major driving force behind this type of cell death.

In addition, during a stroke or other crisis, the problem of glutamate accumulation (primarily caused by the glutamate transport system running out of energy) is rendered even worse, by another factor. Under conditions of severe stress, glial cells (which under normal and healthy conditions had performed the role of “mopping up” any glutamate molecules that had escaped from the synaptic junctions) can begin releasing their own accumulated stores of glutamate molecules. If this process begins to occur during a crisis such as a stroke, it will further aggravate and increase the excitotoxic damage that will be caused by excess extracellular glutamate.

Thus, it is known that glutamate, which is an essential neurotransmitter, takes on an entirely different role in a crisis such as a stroke, cardiac arrest, severe epileptic seizure, or head trauma. In those types of crises, an essential neurotransmitter becomes a toxin that plays a major role in worsening the extent and severity of excitotoxic brain damage caused by the crisis.

After researchers learned that excessive activation of NMDA receptors plays a major role in causing or aggravating a number of types of medical crises (and may also play an important role in aggravating some neurodegenerative diseases as well), many researchers in pharmaceutical companies and academic centers began developing candidate drugs that can suppress activity at NMDA receptors. The hope was that if these candidate drugs could indeed reduce excitotoxic glutamate activity at NMDA receptors, they could reduce and minimize the severity and extent of brain or spinal cord damage after a stroke or other trauma, or in various neurological disorders.

NMDA antagonist drugs were indeed shown to have impressive and highly promising neuroprotective properties, when tested in animal models that simulate stroke. However, when those drugs were subsequently tested in human clinical trials, they were shown to cause hallucinations and other effects that may mimic psychosis.

Subsequent tests in lab animals then revealed that NMDA antagonist drugs, when administered at high dosages, have serious neurotoxic side effects. Even though they could reduce and minimize excitotoxic damage in a patient suffering a stroke or similar crisis, NMDA antagonist drugs were found to inflict a secondary type of neurotoxic injury to neurons in certain other vulnerable regions of the brain.

At moderate doses of a highly potent NMDA antagonist (such as dizocilpine, also called MK-801, or phencyclidine, also called PCP), the neuronal stress typically is manifested initially in reactions such as vacuole formation (generally caused by vacuous swelling of mitochondria and endoplasmic reticulum) in cerebrocortical neurons, and in expression of so-called “heat shock” proteins (which are created as a cellular response to severe stress; these proteins were first detected when cells were immersed in very hot water, and then rescued). These types of stress reactions usually were reversible if the exposure to MK-801 or PCP was only brief, but these stress responses progressed to irreversible damage and neuronal death, if exposure to the NMDA antagonist was prolonged.

Over the past decade, it has become evident that NMDA antagonist drugs which are potent enough to actually reduce brain damage, following a stroke or other crisis, will also cause unwanted and dangerous neurotoxic and psychotomimetic side effects, if administered at dosages high enough to actually reduce excitotoxic brain damage following a crisis. It has become clear that NMDA antagonist drugs pose enough of a risk of permanent brain damage that they must be carefully controlled.

Two facts can help illustrate these dangers. One fact is this: as of this writing, in February 2003, more than 15 years after the first known and effective NMDA antagonist drugs were identified, the FDA has never yet knowingly approved, for public use and sale, even a single type of drug which was known to have NMDA antagonist properties. The FDA has never knowingly approved any such NMDA antagonist for public use, even when limited to a “hospital use only” or “prescription only” basis under the control and supervision of a physician. The sole exceptions to date that have been approved by the FDA have been strictly limited to clinical trials only, for the express purposes of gathering more data as research continues. Not even the mildest potentially effective NMDA antagonists (such as memantine, which has been the subject of a determined effort lasting for years to obtain FDA approval) have been knowingly approved for public use and sale, by the FDA. Over the past decade, numerous candidate NMDA blocker drugs were entered into human clinical trials, but each and every one of those drugs (with the sole exception of memantine, which is still being studied) were subsequently abandoned.

The second fact which illustrates the dangers and risks of NMDA blocker drugs is this: the NMDA blocker drug called phencyclidine (or PCP) is sold illegally under the street name “angel dust”, and it is one of the most dangerous “recreational” drugs ever created. PCP users experience vivid hallucinations and out-of-body sensations that last for hours, and they typically feel no pain of any sort, while they're high. That combination of a hallucinatory and out-of-body experience, when added to the absence of any normal pain sensations, poses a dangerous and volatile combination, and PCP users often launch into violent and uncontrollable psychotic episodes, and may attack innocent people; PCP users have committed numerous gruesome murders, with no provocation of any sort. PCP once was used widely as an animal anesthetic, by veterinarians, but that use has dropped off sharply, partly because of the dangers PCP is now known to pose to the brains of the animals being treated, and partly because of the growing threat of drug abusers committing burglary, armed robbery, kidnaping, and other crimes in the offices of veterinarians who still store and use phencyclidine.

Ketamine is one of the few NMDA antagonists that is currently being used in human medicine. It was approved, for short-term use as a surgical anesthetic, roughly 40 years ago, before it was known to have any NMDA antagonist properties (that approval by the government was granted, decades before NMDA receptors were even discovered). It is difficult to know whether ketamine would be approved by the FDA or other regulatory agencies in other countries, if it were being submitted for approval today, and one can only speculate on that question. However, despite the concerns over the neurotoxic side effects of NMDA antagonists, three factors apparently have convinced most surgeons and anesthesiologists that ketamine is sufficiently safe for normal use as a surgical anesthetic. First: as can be shown in cell culture tests, it is substantially less potent and aggressive, at binding to and blocking NMDA receptors, than MK-801 or phencyclidine. Second: it is used mainly for relatively brief periods, such as an hour or less, for surgeries such as setting a broken bone, or sewing up a wound (although in some cases it is still used for longer types of surgery). And third: anesthesiologists have realized that ketamine should be coadministered along with a benzodiazepine-type drug, such as diazepam (sold under the trademark VALIUM), which helps suppress unwanted excessive neuronal activity. This co-administration of a sedative-type drug such as diazepam helps reduce the risk and severity of a transient form of disorientation and/or psychosis, commonly called a “ketamine emergence reaction”, which occurs among some surgery patients as they wake up after being treated with ketamine if administered without a second drug such as diazepam.

However, to offset those reassuring factors, it also should be recognized that ketamine has become widely and illegally abused in recent years. It has become popular as a recreational drug of abuse, referred to on the streets and in dance clubs by names such as “Special K”. When used in that manner, it apparently causes euphoric and/or out-of-body sensations, mild to moderate hallucinations, etc.

Because ketamine is by far the most potent NMDA antagonist that is available for use by physicians and anesthesiologists today (dextromethorphan, the cough-suppressing agent, also has a weak level of NMDA antagonist activity, but it is much less potent than ketamine), ketamine has been used on a number of occasions, in small-scale trials, for relief of severe intractable pain. To the best of the Applicants' belief, most such small-scale trials in the prior art have fallen into either of two tightly limited categories.

One category involves use of long-term ketamine on patients in the advanced stages of a terminal disease, who will die fairly soon regardless of what is done to treat their pain. Examples of such treatments using ketamine are described in articles such as Klepstad et al 2001 and Kannan et al 2002. In that type of situation, the use of ketamine is humanitarian, and is designed solely to relieve suffering in a patient who will die soon no matter what is done. Clearly, any concerns about possible neurotoxic side effects in that type of use are irrelevant, and are much less important than controlling the pain in a dying patient who otherwise would be in agony.

The second category of small-scale trials, which have tested ketamine for relief of chronic pain, used dosage regimens that were limited in various ways, such as by making the dosages intermittent rather than continuous. As examples, in the trials carried out by Fitzgibbon et al 2002, a single infusion of ketamine over 24 hours was followed by once-per-night pills; Rabbin et al 2001 reported single intramuscular injections, followed by once-per-night pills; and, Mitchell 2001 reported a series of 21 infusions over a period of four months, which works out to an average of one relatively brief infusion every 6 days. Clearly, those regimens did not approach a dosage regimen as disclosed herein, which used continuous intravenous infusion for a span of multiple days.

The closest prior art known to the Inventors herein is contained in two articles (Eide et al 1994, and Eide et al 1995) by a research team in Norway. The 1994 report compared a single intravenous injection of ketamine (0.15 mg/kg) against a single IV injection of morphine (0.075 mg/kg). The reported results included that ketamine was providing a limited degree of relief in some (but not all) of the pain indices that were being measured, and the report also stated that, “Side effects were observed in all the 8 patients after injection of ketamine and in 6 patients after injection of morphine.” This led to a followup study, involving 5 of the same patients who had responded positively in the tests involving acute injections, in which ketamine, administered by subcutaneous (rather than intravenous) injections over a continuous span of 7 days and nights. Although there was indeed some degree of reduced pain, those researcher concluded that this type of treatment was “associated with intolerable side effects” (quoted from the abstract).

“Safener” Drugs

When evaluating the prior art concerning NMDA antagonist drugs, it should be noted that a number of drugs which have been called “safener” drugs can be administered, along with NMDA antagonist drugs, to reduce or eliminate the neurotoxic side effects that are caused by potent NMDA antagonists (such as MK-801 or phencyclidine), or by large doses of ketamine.

A clear, unambiguous, and readily-evaluated standard to determine whether some drug is or is not a “safener” drug (as that term is used herein) can be generated and applied fairly easily, using rats, in tests that use an NMDA antagonist drug called MK-801. This compound is the maleate salt of dizocilpine, and it is highly potent in blocking activity at NMDA receptors. It also is highly selective for NMDA receptors, and has no known interactions with any other types of neuronal receptors. Therefore, it is widely used and well-known in research on NMDA receptors. When injected intraperitoneally into rats, at one-time dosages of about 0.5 milligram (or greater) of MK-801 per kilogram of rat body weight, it will cause a clear and easily detectable vacuole response in specific regions of the rat brain, including the posterior cingulate and retrosplenial cortices. These types of assays are described in more detail in articles such as Olney et al 1991 and Farber et al 1995.

Accordingly, if a candidate drug can substantially reduce or entirely block the vacuole response, in rat brains, when a dosage of 0.5 mg/kg or greater of MK-801 is administered to the rats, then that candidate drug falls within the definition and boundary of “safener” drugs as used herein.

It should be noted that vacuole formation is generally used as a standard indicator or neurotoxic side effects of NMDA antagonists, since vacuole-containing neurons can be observed and counted fairly easily and inexpensively, by using a light microscope after certain relatively simple fixation and staining steps have been carried out. By contrast, evaluating and quantifying the other types of neurotoxic side effects caused by NMDA antagonist drugs require more time-consuming and expensive procedures and reagents. Several such other types of assays are described in other sources, such as in Example 1 of U.S. Pat. No. 5,877,173 (Olney et al 1999), and can be used if desired in any particular type of animal test.

It should be noted, in passing, that the term “safener” was adopted from a different but analogous practice, used by herbicide manufacturers and farmers. If that practice in the realm of herbicides is understood, it may shed more light on this effort to import the concept of safener technology from a different area of science, into the pharmaceutical realm.

Briefly, a number of safener compounds are known that can be applied to certain specific types of crops; this type of safener application is done by coating the safener compound onto seeds, or by spraying a field of crops with the safener, a week or so before a potent weed-killer is sprayed on the same field of crops. The safener compound will trigger a response in the crops, usually involving overproduction of a certain enzyme that will detoxify a certain type of herbicide. This leads to the crops developing a higher level of tolerance for (and resistance to) a certain class of herbicides. After this type of safener treatment has been applied, a certain type of weed-killer herbicide can be applied to the “safened” crops at a heavier dosage, which can achieve greater and more effective killing of any weeds without damaging the crop plants that were protected by the safener.

Nearly 20 safener-herbicide combinations are used commercially, and those can be located fairly easily in an Internet search. As an example, safeners such as dichlormid or benoxacor can be used, in corn and sorghum, to increase the resistance of those particular plant types to acetanilide or thiocarbamate herbicides.

That digression into herbicide terminology was included in this analysis, because the safener approach is a potentially very powerful, effective, and useful approach, but apparently it has been ignored, prior to this invention, both by the pharmaceutical industry, and by government agencies that regulate pharmaceuticals. That is a shame, because herbicide scientists have proved beyond question or doubt that proper use of safeners can indeed protect valuable crops against severe damage that would otherwise be caused by herbicidal toxins.

In a different but analogous manner, safener drugs as disclosed and used herein can protect a mammalian CNS (or at least certain vulnerable portions thereof) against the neurotoxic side effects of NMDA antagonist drugs. This can allow the safe and effective use of NMDA antagonist drugs, at relatively high and prolonged dosages that otherwise could not be tolerated by patients, or approved by government agencies.

Drugs that have been shown to be safener drugs (using the benchmark test specified above, this includes drugs that have been shown to substantially reduce or prevent the neurotoxic side effects of MK-801, in tests on rats) can be divided into several categories, depending on which neurotransmitter system they affect. Briefly, they include:
(1) drugs that suppress activity at the muscarinic m3 class of acetylcholine receptors, as described in U.S. Pat. No. 5,034,400 (Olney 1991);
(2) “direct-acting” GABA agonist drugs, as described in U.S. Pat. No. 5,474,990 (Olney 1995);
(3) alpha-2 adrenergic agonists, as described in U.S. Pat. No. 5,605,911 (Olney et al 1997);
(4) drugs that suppress activity at non-NMDA (kainate and/or AMPA) receptors, as described in U.S. Pat. No. 5,767,130 (Olney 1998); and,
(5) drugs that activate the 5HT-2A (but not the 5HT-2C) subclass of serotonin receptors as described in U.S. Pat. No. 5,902,815 (Olney et al 1999).

However, in evaluating this current invention, it should be noted and recognized that even though these “safener” drugs began to be disclosed more than 10 years ago, not a single pharmaceutical company anywhere in the world has ever chosen to develop, obtain approval for, and market, a combination of an NMDA antagonist drug together with a safener agent. Even though neurology researchers have known for at least a decade that: (i) excitatory activity at NMDA receptors plays a major role in pain signalling and transmission; (ii) NMDA antagonist drugs which can block that type of excitatory activity offer a very promising way to reduce and control chronic and/or neuropathic pain, and (iii) various “safener” drugs are available that can reduce or entirely prevent the neurotoxic side effects of NMDA antagonist drugs, no person, research group, or company (prior to the Inventors herein) has ever figured out how to use any NMDA antagonist drug as a way to safely and effectively control and treat chronic and/or neuropathic pain, in a way that could be approved by the proper government agencies and then commercialized for public use.

It also should be noted that several relatively mild NMDA antagonist drugs are known, which appear to have inherent safening activity, presumably due to simultaneous activity at some other type of neuronal receptor. Those compounds include ibogaine (and a related analog called ibogamine), which are believed to suppress excitatory activity at sigma receptors (these compounds are discussed in, e.g., Deecher et al 1992 and U.S. Pat. No. 5,925,634, Olney 1999), and two compounds called eliprodil and ifenprodil, which are believed to increase inhibitory activity at sigma receptors, and which may also be active at serotonin receptors (these compounds are discussed in, e.g., Carter et al 1988, Sanger et al 1995, and Grimwood et al 2000). However, none of those drugs have been used in any way that even remotely resembles the treatments disclosed herein.

Accordingly, one object of this invention is to disclose that, if an FDA-approved NMDA blocker drug such as ketamine is administered at a “maximum tolerated” dosage (which must be carefully titered, individually, for each patient) over a sustained period of time (such as preferably two or more consecutive days), into a patient who is suffering from chronic and/or neuropathic pain, the NMDA blocker drug apparently can create a profound and long-lasting reduction in the severity of the chronic and/or neuropathic pain, in at least some patients.

Another object of this invention is to disclose that if an NMDA blocker drug such as ketamine is administered at or near a maximum tolerated dosage (preferably titrated for each patient) over a sustained period of time, along with the addition of a “safener” drug that can prevent the neurotoxic side effects that ketamine can cause at very high dosages, then the combined drug regimen may both: (i) provide a more effective reduction in chronic and/or neuropathic pain, and (ii) reduce or eliminate any risk of adverse side effects.

Another object of this invention is to disclose that if ketamine is administered at a maximum tolerated dosage over a sustained period of time along with an alpha-2 adrenergic agonist drug (such as clonidine), the combined drug regimen can provide more effective and longer-lasting reductions in the severity of chronic and/or neuropathic pain, in at least some patients.

Another object of this invention is to disclose that if an NMDA blocker drug such as ketamine is administered at a maximum tolerated dosage over a sustained period of time along with an alpha-2 adrenergic agonist drug such as clonidine, the combined drug regimen can provide more effective and prolonged reductions in the severity of chronic and/or neuropathic pain, in at least some patients.

Yet another object of this invention is to disclose that a safener drug can provide an additional margin of safety, if prolonged administration of an NMDA antagonist such as ketamine is used to try to provide lasting relief from chronic or neuropathic pain.

Yet another object of this invention is to disclose that prolonged administration as disclosed herein of an NMDA blocker drug such as ketamine, preferably in conjunction with a second drug such as an alpha-2 adrenergic agonist drug, to a patient who is suffering from an unwanted neurological condition (such as tolerance for, dependence on, or addiction to, a drug, nicotine, or alcohol), can help at least some patients establish a higher, better, and more secure level of control over the unwanted neurological condition, in a manner analogous to activating a “reset button” or “return to default settings” control on a computer or programmable machine. It is also believed that this type of neurological intervention (especially if used in conjunction with counseling, therapy, etc.), in addition to helping treat patients for various types of addictions or dependencies, can help at least some patients establish better and more reliable levels of control over various other neurological and/or psychological or emotional disorders as well, such as (for example) some types of obsessive-compulsive disorders, some types of bipolar disorder, clinical depression and other affective disorders, and/or emotional disorders, some types of psychological addictions or cravings, etc.

These and other objects of the invention will become more apparent through the following summary, drawing, and description.

### SUMMARY OF THE INVENTION

Methods are disclosed for prolonged and continuous intravenous infusion of an NMDA receptor-blocking drug such as ketamine, which has been approved for sale and use in humans for short-term use as a surgical anesthetic, into patients who suffer from neuropathic or other types of chronic intractable pain, in order to provide long-lasting reduction in the pain, even after the NMDA antagonist drug is discontinued. In this treatment, which preferably lasts for several days, the patient does not lose consciousness except during natural sleep, and the dosage must be individually titered to achieve the maximum tolerated dosage for each particular patient.

Unless the NMDA drug has inherent safener activity of its own, this treatment also requires the co-administration of at least one safener drug, which has been shown to reduce the neurotoxic side effects of potent NMDA antagonists (which can be easily demonstrated in animal tests). A properly selected safener drug preferably should also potentiate and increase the pain-relieving efficacy of the overall treatment; one such class of safener agents include alpha-2 adrenergic agonists, such as clonidine.

Co-administration of a magnesium salt is also preferred, and apparently can increase the pain-relieving efficacy of this treatment in at least some cases. As used herein, the term “salt” includes any compound or complex that releases substantial quantities of free magnesium ions (Mg⁺⁺) when dissolved in an aqueous solution.

Several highly successful clinical treatments are disclosed, in which patients who had suffered for years from an especially severe, chronic, and intractable pain condition, were able to emerge from this new treatment, apparently cured completely. The condition these patients suffered is referred to by either of two terms, which are overlapping and usually interchangeable. One of these terms is “Reflex Sympathetic Dystrophy” (abbreviated as RSD), and the other is “Complex Regional Pain Syndrome, Type 1” (usually abbreviated as CRPS-1 or CRPS-I). This is an extremely difficult and intractable problem, which causes debilitating atrophy and wasting of the muscles and other tissues involved.

The only two CRPS-1 (RSD) patients whose results were less than fully successful were subsequently discovered to have lower-than-targeted concentrations of ketamine in their blood plasma. The desired concentrations are at least about 250, and preferably about 350, nanograms of ketamine per milliliter of blood plasma, and the two patients who did not have fully satisfactory outcomes had substantially lower concentrations, despite the titering efforts that were carried out on each of those patients, as described below. The low ketamine plasma concentrations seen in those two patients is presumed and believed to be due to variations in a set of liver enzymes generally known as “cytochrome oxidase” and/or “P450” enzymes. These liver enzymes play a major role in degrading and metabolizing any non-natural molecules that are circulating in the bloodstream, including numerous types of drugs, and humans are known to have wide variations in their cytochrome oxidase enzyme activity levels. Accordingly, any liver-dependent variations in blood concentrations in certain individuals can be overcome, if analytical data indicating ketamine concentration in their blood plasma can be determined within a span of about 12 to 36 hours. As of this writing, that service is not available anywhere, because there has never been a need for such information, and blood concentration data for ketamine could be obtained only after a waiting period of roughly 2 weeks; this meant that the data could not be used in a timely or effective manner, since the continuous infusion period had already ended, usually after about 3 to 5 days. Because this invention discloses an important and highly valuable need for rapid ketamine concentration analyses, this invention also discloses and anticipates the development of such rapid blood analyses, as an independent commercial operation, and as one of the steps that will be used to carry out the invention disclosed herein.

It is believed that this type of treatment can effectively give a patient's central nervous system a chance to re-establish an “altered homeostatic equilibrium”, at a new and different yet stable and sustainable plateau (or “set point”). As such, it is believed that this treatment, in addition to providing a highly effective method for treating severe intractable pain, is also likely to be useful in treating other types of neurological disorders, including the following: (i) addictions or intense cravings for alcohol, nicotine, illegal or narcotic drugs, and similar substances; (ii) neurological disorders that are manifested with or through emotional or behavioral aspects, such as obsessive-compulsive disorders, depression and other affective disorders, severe phobias, compulsive gambling, sexual disorders, and possibly bipolar or other disorders; (iii) progressive neurodegenerative diseases, such as Parkinson's disease, Huntington's disease, amyotrophic lateral sclerosis, dementias such as Alzheimer's disease; etc.; and, (iv) various other conditions that have a neurological component, such as fibromyalgia.

### BRIEF DESCRIPTION OF THE DRAWING

FIG. 1 depicts both the ketamine dosage (dotted line with square data points) and the reported pain levels (solid line with triangles) for patient described in Example 2.

### DETAILED DESCRIPTION

The method and invention disclosed herein arise from a series of highly successful results that were observed when human patients, who had suffered from intractable neuropathic pain for years, were treated with sustained infusions of ketamine.

Most of these patients (except for several who suffered from shingles) suffered from an especially difficult and debilitating pain syndrome, which can be referred to by either of two terms, which are overlapping and usually interchangeable. One of these terms is “Reflex Sympathetic Dystrophy” (abbreviated as RSD), and the other is “Complex Regional Pain Syndrome, Type 1” (usually abbreviated as CRPS-1 or CRPS-I). These classifications are described in publications such as Merskey et al 1994, Harden et al 2001, and Schwartzman et al 2002.

Briefly, RSD is diagnosed by the presence of five major features: pain, swelling, autonomic dysregulation, movement disorders, and atrophy and dystrophy. The last feature (atrophy and dystrophy) deserves special attention, because this feature indicates that RSD causes the wasting-away of muscle and other tissue. Accordingly, it is an extremely serious and debilitating disease, and it often forces patients to quit working and begin requiring insurance, disability, and/or unemployment benefits.

The “Complex Regional Pain Syndrome” nomenclature was adopted more recently, and it uses somewhat different approaches to defining CRPS, but the overall result is that most cases that are properly diagnosed as RSD can also be properly diagnosed as CRPS-type 1. The major difference between the CRPS-1 and CRPS-2 categories is that cases in the CRPS-2 category usually arise as a result of a clear and discernible traumatic injury, such as an automobile or bicycling accident, a bad fall, shooting or stabbing, etc. By contrast, cases in the CRPS-1 category do not arise as a result of any single known traumatic event, and instead arise due to some form of neuropathology, rather than a major trauma, in which some type of genetic condition, minor triggering event, circulatory disorder, and/or other or similar triggering factor(s) set in motion some type of internal sequence of neuropathological events and developments, which eventually culminate in an extremely painful form of neuropathic pain.

This is indeed what happens in most cases of RSD; most cases of RSD do not arise from any known injury that causes an identifiable nerve lesion, and therefore these cases fall within the CRPS-1 category.

This is just a brief overview, and additional information on these pain syndromes is contained in numerous published articles and books, including Merskey et al 1994, Harden et al 2001, and Schwartzman et al 2002.

Some of the patients described in Examples 2 and 3 were treated by slightly differing drug regimens, which were honed and developed over time to increase their efficacy, as the results of each individual treatment in the series became known and suggested further enhancements for use in subsequent treatments. The drug regimen described in the following paragraphs (before the examples) evolved and emerged from the results seen during these initial treatments (which are described in the examples), and the protocol described below is believed to be the best approach that has been created to date.

These initial treatments were all carried out in hospitals, with each patient under continuous supervision and monitoring, following standard hospital practices. In general, any patient being treated should be in a relatively quiet private room, to minimize any demands, distractions, and intrusions that might be imposed on the patient during the treatment. The patient can be allowed to have as few or as many visitors as he or she desires, to suit his or her own comfort level.

All patients that were treated during these initial clinical trials suffered from severe chronic and intractable pain, but were otherwise regarded as having low to moderate risks when evaluated as candidates for anesthesia. A standardized scale for risk evaluation has been developed by the American Society of Anesthesiologists, and is described in any medical textbook on anesthesiologists. In this scale, a “1” ranking indicates no known or apparent risk factors, a “5” ranking indicates a severely compromised patient who is under grave risk if subjected to general anesthesia, and the intermediate numbers represent low (2), moderate (3), and high (4) risk factors and rankings. In order to prevent the results and data from the initial treatments from being unjeopardized by uncontrollable variables, these initial treatments were limited to patients in the two lowest-risk categories (i.e., with a ranking of 1 or 2 on the ASA scale). However, subsequent treatments and clinical trials can be performed on patients with higher risk rankings, so long as those patients are fully informed about the risks they are undertaking, and give informed consent.

During a treatment, the patient's blood pressure, heartbeat rate, respiratory rate, and pulse oximetry should all be monitored. Since continuous strip-charts tend to annoy patients, adequate monitoring generally can be done every 1 to 2 hours during the “ramping up” stage while ketamine dosages are being evaluated and changed, and every 2 to 4 hours after the maximum dosage has been reached. During the first night of treatment, when the effects of the drugs on a patient's blood pressure pose the most concern, the patients treated to date were awakened once, at roughly 4 am, for a blood pressure reading. However, on subsequent nights, the nurses were given more flexibility, and they generally should be encouraged to not wake up a patient who is sleeping.

Intravenous infusion is regarded as the preferable mode of administration, since (i) it can avoid the types of adverse skin-related side effects that were reported in Eide et al 1995, and (ii) it can also maximize the effects that can be exerted by the ketamine before it is metabolically degraded into metabolites such as norketamine, which is only about 25% as effective as ketamine in reducing pain signals. Alternate modes of administration may be discovered to be effective, for some types of pain treatments, after sufficient clinical trial data are gathered in the future; however, any initial clinical trials preferably should use intravenous infusion, while the boundaries and limits of this type of treatment are being evaluated.

It also should be noted that terms such as “sustained” and “continuous” are used and intended broadly, and generally include any mode or administration that is designed to sustain concentrations of an NMDA antagonist drug, in circulating blood, at levels that will be remain high, rather than forming intermittent peaks. This does not require absolutely continuous and uninterrupted infusion, 24 hours per day, with no interruptions of any sort. However, it should be recognized that, to achieve the desired effects as disclosed herein, “sustained” and “continuous” administration (especially by intravenous infusion) is strongly preferable, and can achieve better results than administration that would be regarded as periodic or intermittent.

Administration of Clonidine or other Safener Drugs

Roughly 2 hours before ketamine infusion begins, the patient should be given an oral dose of a “safener drug” that can reduce or eliminate the risks of any neurotoxic side effects that might otherwise be caused by continuous prolonged administration of the ketamine. A preferred class of safener drugs, which appears to significantly enhance the efficacy of ketamine in providing long-term relief from pain when used as disclosed herein, comprises alpha-2 adrenergic agonist drugs, such as clonidine. As an additional benefit, clonidine (and various other alpha-2 adrenergic agonist drugs) can also help reduce blood pressure. This is useful, when used in conjunction with ketamine, since elevated blood pressure can be a side effect of ketamine, due to certain activities involving the sympathetic nervous system and the release of catecholamines.

In the treatments disclosed herein, clonidine tablets containing 0.1 or 0.2 mg were administered orally, every 8 to 12 hours, with good results. Depending on the assessment of the treating physician, this dosage can be increased after ketamine infusion has commenced, so long as blood pressure monitoring does not indicate a worrisome drop in blood pressure. The transdermal form of clonidine (i.e., using skin patches) has also been used, with good results.

Clonidine administration should continue as long as ketamine is being administered. After ketamine is terminated, the clonidine should be tapered off, generally over a period of about 1 to 3 days, unless the patient must also go through a sustained period of withdrawing from morphine, OXYCONTIN™, or other high-strength pain-killing drugs (such drugs are widely used among patients who suffer from intractable pain). In the treatments done to date, dosages of morphine or OXYCONTIN were typically reduced by about 25% on the second day of treatment, and they were reduced by another 25% during the next few days, while ketamine treatment continued. The cessation of the remaining dosage of morphine or OXYCONTIN was then usually carried out over a 3-week period, after the ketamine treatment had ended.

If a weaning period is used (during and/or after the ketamine treatment) to help a patient stop taking OXYCONTIN, morphine, or similar narcotic drugs, elevated blood pressures are likely to result from the drug withdrawal response. Accordingly, clonidine can be continued for as long as appropriate to help control that type of elevated blood pressure.

If a particular patient (such as a patient who suffers from low blood pressure, or who suffers from nausea or other serious side effects caused by clonidine) cannot easily withstand clonidine, any of various other candidate safener drugs can be tested for use with that patient, along with ketamine. Candidate agents that may merit evaluation for use with any or all of various categories or subcategories of patients include the following classes of drugs:
(i) other known drugs that stimulate activity at the alpha-2 subclass of adrenergic receptors. This includes a number of known drugs, including (for example) iodoclonidine, guanabenz, xylazine, medetomidine, tizanidine, rilmenidine, alpha-methyldopa, and alpha-methylnoradrenaline (see, e.g., Goodman and Gilman 1990 at page 308; Ruffolo et al 1993 at page 264; and Doze et al 1989 at page 75). These drugs tend to have moderately high levels of activity at both the alpha-1 and alpha-2 receptor subtypes, and it should be noted that a number of other candidate drugs have been developed which are believed to have more highly selective affinity for alpha-2 receptors than for alpha-1 receptors, when compared to clonidine. The more highly selective alpha-2 adrenergic agonists include guanfacine, dexmedetomidine, and azepexole, and may also include lofexidine and various other recently discovered alpha-2 agonists as well. Adrenergic receptor selectivity for any candidate adrenergic agonist drug can be evaluated by using competitive binding assays, using methods known to those skilled in the art (and using genetically-transformed cells that express only one of the alpha-adrenergic receptor subtypes, if desired).
(ii) drugs which act as agonists at gamma-amino-butyric-acid (GABA) receptors. These drugs can generally be divided into two subclasses: (1) indirect GABA agonists, which work by increasing the effects of naturally occurring GABA at GABA receptors (these drugs include the benzodiazepines, such as diazepam, which is sold under the trademark VALIUM); and, (2) direct GABA agonists, which function by direct interactions with GABA receptors, regardless of whether any naturally-occurring GABA is present. Because direct GABA agonists do not require the presence of naturally-occurring GABA at the GABA receptor sites, they tend to be more effective, potent, and controllable for this type of use, and they are generally preferred, although they must be limited to carefully controlled dosages, to avoid rendering a patient unconscious. Examples of direct GABA agonists that can be evaluated for such use include propofol (which is sold in an injectable emulsion formulation, under the trademark DIPRIVAN), and various analogs and derivatives of propofol, as described in articles such as Trapani et al 1998. Other examples of direct GABA agonists include various barbiturate drugs, including pentobarbital, secobarbital and thiamylal;
(iii) drugs that suppress activity at the muscarinic m3 subclass of acetylcholine receptors; such drugs include scopolamine, atropine, benztropine, trihexyphenidyl, biperiden, procyclidine, benactyzine, and diphenhydramine;
(iv) drugs that suppress activity at the kainate and/or AMPA subclasses of glutamate receptors, such as a drug called NBQX (described in Sheardown et al 1990 and PCT application WO-92/11012), and a drug called GYKI 52466 (described in Tarnawa et al 1990); and,
(v) drugs that activate the 5HT-2A class of serotonin receptors, but that do not also show substantial affinity for the 5HT-2C class of serotonin receptors. Such drugs are believed to include lisuride (sold in Europe under trademarks such as Cuvalit, Dopergin, Eunal, and Lysenyl, to treat migraine headaches and Parkinson's disease, and to help women stop lactating after nursing a baby), and possibly a drug called MDL 100,907 (Schmidt et al 1995).
   Magnesium Administration

Beginning roughly 2 hours before the ketamine infusion is commenced (this can be done conveniently at the same time when the first clonidine tablet is taken), the patient should also be administered magnesium, preferably by intravenous infusion of a water-soluble salt, such as magnesium sulfate or chloride. This can be done by dissolving 2 grams of MgSO₄ in 250 milliliters of 5% dextrose in water (abbreviated as D5W) and infusing the liquid into the patient over a period of about 2 hours.

A second infusion of 2 grams of MgSO₄ should be administered roughly 24 hours later. This can be repeated each day, if desired, if the patient has not yet achieved complete cessation of the chronic pain; however, in the treatments completed to date, it has never been provided more than 3 times.

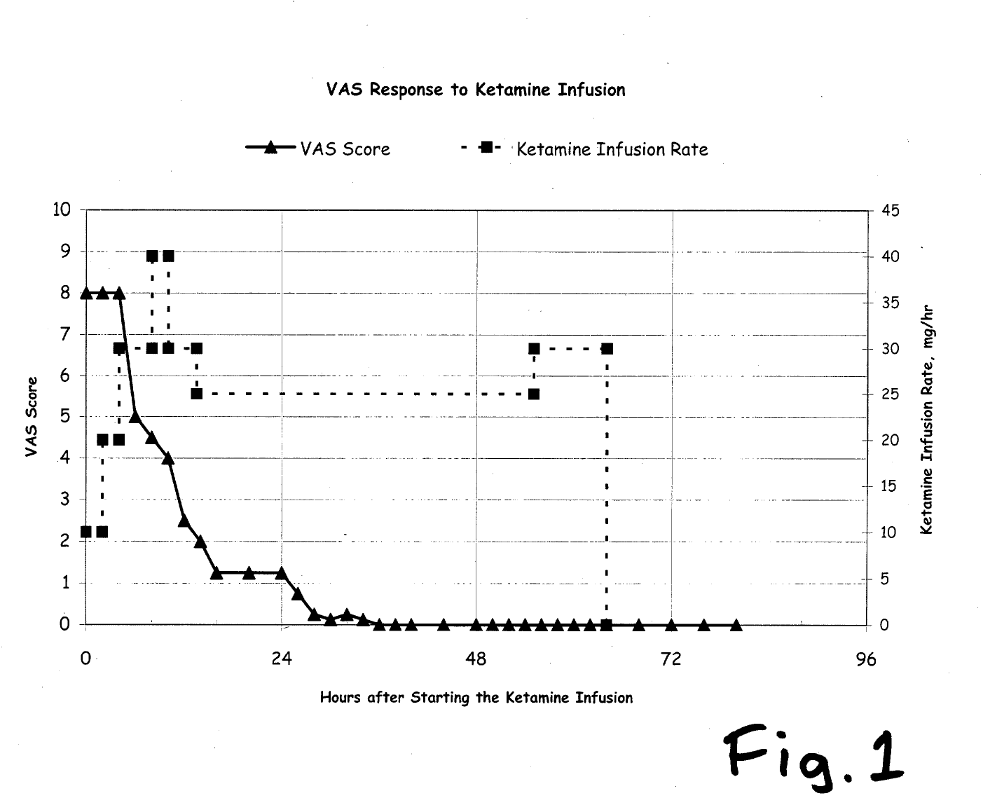

It should be noted that in FIG. 1, which shows a patient's reported pain levels as a function of time, the final drop-off of reported pain levels from a plateau that had been reached, to zero, coincided with the infusion of a final dosage of MgSO₄. That patient's pain level had dropped from 8 (the baseline level when ketamine treatment began) to about 1.3, after 24 hours. However, it had leveled off at about 1.3, and it was not going down any more, despite the fact that more ketamine was being administered continuously. However, when a second MgSO₄ infusion began, commencing at 24 hours, the pain level began dropping once again, and within about 3 hours it had decreased completely to zero.

Ketamine Infusion: Titering and Blood Concentrations

As soon as the initial magnesium infusion (which takes about 2 hours) has been completed, the patient is ready for the ketamine infusion to begin. In the treatments described herein, since these were done without being able to rapidly determine ketamine concentrations in blood, each patient was individually “titered”, by testing a series of gradually increasing dosages until a “maximum tolerated dosage” was reached and determined.

This typically was done by starting each patient at an initial dosage of 10 mg per hour, and then evaluating the patient every 2 hours after that, to see whether the dosage should be increased by another 5 or 10 mg/hr. When a patient began showing or reporting substantial CNS effects (such as feeling drunk, slurring of speech, semi-hallucinatory thoughts or visions, etc.), it was presumed that the maximum tolerated dosage had been reached or slightly passed, and the dosage was decreased slightly (such as by 5 or 10 mg/hr, depending on how well the reported pain levels were decreasing).

Typical maximum tolerated dosages, for patients treated to date, ranged from 20 to 45 mg/hour. These final dosages did not appear to be dependent on a patient's body weight; instead, their variability in different patients were believed to depend more heavily on the activity levels of a class of enzymes in the liver, known as “cytochrome oxidase” and/or “P450” enzymes, which are known to degrade and metabolize drugs. Various drugs are known which can inhibit some of those enzymes, as described in articles such as Inaba et al 1985, and in various patents issued to Richard Smith on combinations of quinidine and dextromethorphan. If desired, any of those enzyme-inhibiting drugs can be evaluated for potential use in suppressing one or more liver enzymes in a way that may help sustain and prolong elevated ketamine concentrations during an infusion regimen as disclosed herein.

Blood samples were taken from each patient during his or her infusion regimen, and those samples were shipped to an analytical laboratory (National Medical Services Company, in Willow Grove, Pa.) to have their ketamine concentrations analyzed. Those results indicated that blood plasma concentrations greater than about 250 (and preferably about 300 to 400) nanograms (ng) of ketamine per milliliter (ml) of plasma appeared to provide the best results. The blood data from two of those patients (described in Example 2, below) later revealed that those two patients did not reach or even closely approach the 300 ng/ml level. This was noteworthy, since those two patients did not receive complete and long-lasting relief from their pain. In view of those data, it is believed that blood plasma concentrations of at least about 200 ng/ml (and preferably at least about 250 to about 350 ng/ml) should be regarded as target levels for effective treatments.

In the treatments described herein, blood concentration data did not become available, for any particular patient, until two weeks after that patient's blood sample was shipped to a lab for analysis. That two-week delay was reasonable, in view of the fact that there has never before been any known utility for determining ketamine concentrations in blood any faster than that. However, in view of the disclosures herein, a new need and utility has now been established, which will benefit greatly from rapid analysis of ketamine levels in circulating blood.

Accordingly, the inventors herein have taken steps to work with a qualified analytical company, in order to encourage that company to analyze blood samples and provide ketamine concentration data within less than a day after receipt of a sample. It is believed that rapid analyses (which can be completed within a span of generally less than about 24 hours, after a blood sample has been received by a lab) can be developed by those skilled in the art, using methods selected from a range of known candidate methods (such as high-performance liquid chromatography, differential permeation through a gel or other semi-permeable media, immunosorbent assays using monoclonal antibodies or other reagents that will bind to or react with ketamine, etc.).

That type of rapid analysis, for ketamine concentrations in blood, is regarded as a distinct but parallel invention, and the use of blood concentration data from a particular patient who is being treated, during the treatment, is included as one of the preferred steps in some of the embodiments disclosed and claimed herein.

During the first day, while a patient is adjusting to the combination of clonidine, magnesium, and ketamine, the patient tends to feel a bit unsteady and dizzy, so they generally stay in bed, and get help to go to bathroom. After roughly 24 hrs, most patients adapted to the CNS effects of the drugs, and developed a level of motor control tolerance, and they were able to get up and amble around, usually pulling around their own intravenous stand through a quiet courtyard (which was usually a very pleasant and relaxing activity, since it was their first pain-free stroll in a long time).

After a patient has been pain-free for roughly 24 hours, the ketamine dosage can be increased slightly, for a period such as 6 to 12 hours. This is shown in FIG. 1 by the elevated plateau of ketamine dosage lasting from about hour 54 through about hour 62. This “final bump” was used to “drive home” or “nail down” the treatment, in a manner which would help ensure that the effects of the treatment would last longer, by providing a cushion, or margin of safety. To use yet another analogy, the purpose and goal of any “final bump”, and of prolonging ketamine treatment for another day or two after reported pain levels have dropped to zero, is to extinguish or at least minimize any “glowing embers” that might still be burning, and that would pose a threat of turning into open flames once again if the wind picks up.

In the treatments done to date, when blood concentration data were not available until more than a week after a treatment had been completed, it was generally presumed that if pain levels had dropped to a stubborn and persistent plateau (such as about 5 to 10% of baseline levels), and had stayed there for about 24 to 48 hours without going any lower despite receiving the entire set of all three drugs, then the treating physician decided it was time to quit. In future treatments or trials, if blood concentration data become rapidly available during a treatment, the decision by the treating physician as to when the treatment should be terminated can be made in view of the additional data provided by the blood data.

In the treatments done to date, gradual tapering off of the ketamine infusion was not deemed to be necessary; however, that approach can be used, if desired, in any case, under the judgment and control of the treating physician.

As mentioned above, ongoing clonidine administration was continued in most cases done to date, in order to help patients control their blood pressures while they subsequently went through a process of weaning themselves from morphine, OXYCONTIN, or other powerful pain-killing drugs.

Clearly, at this very early stage of clinical treatments, a number of potentially important contributing elements were involved, and their roles should be isolated and analyzed more carefully, as more data are gathered. Potentially significant factors which deserve further analysis include:

1. The method, dosage, and other factors of the ketamine delivery. In the treatments disclosed herein, a relatively slow build-up of ketamine, by intravenous infusion, led to a maximum tolerated dosage, which then continued for several days. This had not been done previously, in any published reports of which the Applicants are aware. It also should be recognized that various non-intravenous modes of administration are also possible, each of which is likely to require substantially different dosages. These include: (i) oral ingestion of formulations such as tablets, capsules, syrups, etc.; and, (2) transmembrane routes, such as nasal sprays (which can be supplemented by mucoadherents, such as chitosan or various polysaccharide colloids, as discussed in articles such as Schipper et al 1999 and Janes et al 2001) and skin patches (which can be bolstered by agents that can increase tissue permeation, such as dimethyl sulfoxide). Any of these or any other known routes of administration can be evaluated through routine experimentation, which generally should focus primarily on blood concentrations as a function of a particular treatment dosage and route. These types of analysis will become substantially easier and more reliable if rapid analyses of ketamine in blood are developed and made available.

2. The duration of the ketamine administration. Previous attempts by others to use ketamine for treating chronic pain involved only short-term infusions, for a period of only a few hours. By contrast, some of the treatments disclosed herein indicated that pain relief did not become evident until after about 2 days of steady infusion. Prolonged subcutaneous infusions of ketamine were tested in the mid-1990's, as reported in Eide et al 1995, summarized above; however, subcutaneous infusions must be infused so slowly that much of the ketamine is likely to be metabolized to norketamine before it can exert its effects, and Eide et al reported that their treatment was “associated with intolerable side effects” (quoted from their abstract).

3. The potential roles and effects of a purified or enriched stereoisomer of ketamine. Very briefly, stereoisomers occur when a single carbon atom (called a “chiral” carbon atom) has four different atoms or groups bonded to it. When that happens, the resulting molecule can have either of two different spatial arrangements. For complex reasons, these can generally be referred to as the “+” and “−” stereoisomers, or as the “D” and “L” isomers, or as the “R” and “S” isomers, depending on (i) how the different groups are organized about the chiral carbon atom, and, (ii) the direction in which polarized light will be rotated, if polarized light is passed through a liquid suspension of the enriched or purified stereoisomer. Currently, a random (or “racemic”) mixture that contains both the “R” and “S” stereoisomers of ketamine in roughly equal concentrations is commonly used for surgical anesthesia, and is approved for such use in the U.S. by the Food and Drug Administration. However, a purified form of the “S” isomer has become available, in Europe, for medical testing. This compound is sold under the name “Ketanest-S” by a German company, and it cannot be imported into America without specific permission as an investigational new drug. Based on cell culture tests, animal tests, and early human clinical trials done to date, the “S” isomer is not believed to have particularly better efficacy than a racemic mixture, in carrying out this invention. Nevertheless, that isomer is available to qualified researchers, and it can be further evaluated for use as described herein, if desired. If the results provided by a pure or enriched stereoisomer appear to be better than the results provided by racemic mixtures, then the pure or enriched stereoisomer can be used in subsequent treatments as disclosed herein.

4. The additional roles of an alpha-2 adrenergic agonist (or other type of safener drug), in modulating the upper tolerated dosage for ketamine, in reducing the risk of neurotoxic damage caused by long-term infusion of ketamine, and in increasing the level and duration of pain relief provided by the ketamine-clonidine combination, compared to the extent and duration of relief provided by ketamine alone.

5. The role of magnesium (and other transition metals that are present in the body) in accelerating or increasing the pain relief response to ketamine. It is known that magnesium plays a significant role in suppressing ion flow through NMDA receptor channels. This effect pulls in the same direction as the suppression of ion flow through the same NMDA receptor channels, by ketamine. Since improved pain relief has been reported fairly quickly after magnesium was administered to a patient in one of the treatments (this type of response is graphically illustrated, in FIG. 1, by the drop in the pain plateau at the 24 hour mark, which began when a second bolus of MgSO₄ began to be infused into the patient), the magnesium is believed to have played at least some contributory role in the relief process. If desired, this factor can be isolated and further evaluated in future treatments and trials.

6. The role of various liver enzymes in degrading ketamine or any other NMDA antagonist drug that may be of interest, and the ability of one or more adjunctive drugs to inhibit such enzyme(s) and thereby increase and prolong the concentration of the NMDA antagonist drug in circulating blood.

7. The efficacy of candidate NMDA antagonists that are believed to have inherent safening activity (such compounds may include ibogaine, ibogamine, eliprodil, and ifenprodil, as mentioned in the Background section, and various analogs and derivatives thereof) in carrying out the treatments of this invention, in ways that might eliminate the need for an additional safener drug.

If desired, these and any other factors, modifications, and alternate candidate NMDA antagonists or safener drugs can be evaluated, in future clinical trials, using no more than routine experimentation, using procedures such as disclosed herein or as otherwise known to those skilled in the art.

Pre-Mixed NMDA Antagonists and Safener Drugs

It is believed by the inventors herein that pharmaceutical mixtures containing a safener drug (such as an alpha-2 adrenergic agonist) that has already been mixed with ketamine (or any other NMDA receptor antagonist drug) would provide a better and safer product, and a better and safer mode of administration of NMDA receptor antagonist drugs, compared to NMDA receptor antagonist drugs that do not also contain any safener compounds. The regulatory agencies that must evaluate and approve human pharmaceuticals in various nations of the world (such as the Food and Drug Administration, in the U.S.) are quite aware of the risk of neurotoxic brain damage by NMDA receptor antagonist drugs, as evidenced by the fact that they have never knowingly approved any NMDA receptor antagonist drug for any human use, despite the well-known potential of those drugs for reducing brain damage after a stroke or cardiac arrest, and in various other medical crises and conditions. There also can be no doubt that, in at least some nations (including the U.S.), there is a severe shortage of skilled nurses and certain other classes of health-care professionals, who face increased risks of making innocent mistakes, each time they are required to administer yet another drug to a patient who is already receiving multiple drugs. In addition, there can be no doubt that NMDA antagonist drugs (notably including ketamine, which is sold illegally on the street and in dance clubs, under the nickname “Special K”) are widely and illegally abused, by illicit drug abusers, in ways that may well be causing permanent brain damage in those users and abusers.

Therefore, pre-mixed combinations of an NMDA receptor antagonist drug (such as ketamine) which have a safener drug already added to the mixture (so that the safener compound cannot be accidentally overlooked or omitted from a patient's intended treatment at a busy hospital or clinic, and so that there will be lower risks of illegal abuse of such drugs) may be able to gain approval for public sale and use, from the necessary agencies, more readily and easily than an NMDA receptor antagonist drug by itself with no safener compound added to it.

Accordingly, the disclosures herein highlight and emphasize the need for combinations of ketamine (or other NMDA receptor antagonist drugs) that have been already mixed (prior to sale) with additional agents that will provide safening activity for the NMDA receptor antagonists. The preferred dosages and ratios for such agents can be determined through routine experimentation (focusing primarily on blood concentrations in human volunteers), for any particular route of administration, and for any combination of a particular selected NMDA receptor antagonist drug and a particular selected safener compound.

Additional details for the patients that have been treated to date in the United States are provided below, in Examples 2 and 3. As briefly mentioned in Example 1, several additional patients were treated prior to that, in Australia, using ketamine only, without any additional clonidine or magnesium. Those results were generally good, in the sense that they were substantially better than could be achieved by any other treatments that were known prior to the creation of this invention. However, those initial results using ketamine alone have been substantially improved by the inclusion of clonidine and magnesium as part of the treatment along with ketamine.

All patients described in Example 2 suffered from a condition that was known until recently as reflex sympathetic dystrophy (RSD), and that recently has been reclassified as “complex regional pain syndrome, type 1” (abbreviated as CRPS-1). This is a complex, intractable, and debilitating medical condition that is well-known to physicians who specialize in treating chronic pain. This condition typically includes at least one, usually more than one, and frequently all three of the following symptoms: (1) burning pain; (2) mechanical allodynia (which indicates that if something touches the skin, in a way that normally would not cause pain, it will provoke a pain response in someone suffering from this condition); and, (3) deep aching pains that feel as though they're emanating from the bones and deep muscles. There are also other symptoms that may occur with RSD, including skin color changes, dystrophic changes of the skin, hair and nails, muscle and joint stiffness, etc. It is not yet certain how this treatment will affect those or other symptoms.

CRPS-1 and RSD are regarded as especially difficult and intractable forms of chronic pain, which cannot be adequately treated using any other known treatments. A number of sufferers and physicians participate in various websites that focus on this type of pain and the community of people who suffer from it; one example is www.rsdhope.org.

Example 3 describes treatment of people who suffered from herpetic shingles, rather than CRPS-1/RSD.

Claims that include Labelling Limitations

This invention also relates to articles of manufacture, comprising: (i) an injectable aqueous formulation containing at least one NMDA antagonist drug, such as ketamine, and preferably also containing at least one safener drug and/or magnesium, enclosed within (ii) a package that maintains sterility of the aqueous formulation and that contains printed information stating that the NMDA antagonist drug is intended for sustained intravenous infusion for several days continuously, at a dosage which must be titrated individually for a specific patient who receives such treatment.

In this type of article of manufacture, which relies upon the printed label as one of the points of novelty, it should be noted and understood that: (i) the printed label is not being relied upon to establish patentability, and the invention as a whole includes, as an essential element, the tangible physical and chemical contents within the package; and, (ii) a number of decisions by the Court of Appeals for the Federal Circuit (and its predecessor) have explicitly stated that if printed matter is merely one item in an otherwise tangible and patentable article of manufacture, then the printed matter cannot be excised or deleted from the item before the Patent Office examines the claim. Those two factors must be considered conjointly with another legal factor: under the laws and regulations that are enforced by the Food and Drug Administration, and that apply to such items of commerce, the contents and the label must be regarded as a single indivisible item of commerce.

### EXAMPLES

### Example 1

### Patients Treated in Australia

About 30 patients have been treated during the span of 1997 through 2002, in Australia, by one of the Inventors herein (Correll), using prolonged infusions of ketamine without any safener drug or magnesium. Those patients enjoyed generally good results, and more than 25 of them were believed to remain generally pain-free (or at least in a state of partial remission which remained substantially better than their condition prior to the treatment), as of the filing date of this application. Those patients have been described in more detail in Correll et al 2004, and more current information on the duration of remission are provided in FIGS. 1 and 2 of that report. That report is not prior art against this invention.

### Example 2

### U.S. Patients with CRPS-Type 1 (RSD)

The first patient treated in the U.S. is described in some detail in Harbut and Correll 2002. That article was written by two of the inventors herein, and it is not conceded to be prior art. The contents of that article are incorporated herein by reference. That patient enjoyed apparently complete remission for about 18 months, but at that time, an original contributing problem recurred, which led to a second and substantially less intense recurrence of her neuropathic pain problem.

Four other patients who had suffering from CRPS type 1 for at least two years were subsequently treated in the U.S. Because of patient privacy concerns, the details of their condition cannot be disclosed herein. Typical treatments for these patients commenced with 2 grams of MgSO₄ (infused in 5% dextrose in water, or D5W) and 1 or 2 tablets of 0.1 mg clonidine, two or three times per day as tolerated, commencing at least an hour prior to the first ketamine infusion. Throughout the treatment, patients continued to receive 1 or 2 tablets of clonidine 2 to 3 times per day, as tolerated, and a second magnesium infusion was performed in some patients at 24 hr. Ketamine was initially infused at 10 mg/hour, and the infusion rate was gradually increased until a patient began to show CNS effects, such as slurring of speech.

One of those four patients has enjoyed apparently complete relief, which appears to be permanent as of the filing date of this application.

Another patient enjoyed apparently complete relief for roughly six months, but then began to suffer from severe health problems unrelated to neuropathic pain, which led to a recurrence of chronic pain. The chart provided in FIG. 1 depicts the treatment of that patient.

Two other patients suffered a return of substantial levels of pain, one to roughly the same starting level that existed prior to treatment, and one to roughly 50% of the pretreatment level. Blood analyses, which did not become available until after their treatments had been finished and terminated, later showed that their blood plasma concentrations of ketamine were generally less than the desired levels that are now regarded as the minimum effective level, presumably due to relatively high rates of metabolism of ketamine by enzymes that function primarily in the liver. Blood from one patient was analyzed for norketamine, a metabolite of ketamine that is only about 25% as effective as ketamine in suppressing activity at NMDA receptors. That analysis indicated a high level of norketamine, which confirmed that certain enzymes in that patient were unusually active in rapidly degrading ketamine.

### Example 3

### Patients with Post-Herpetic Neuralgia (Shingles)

Several patients were also treated who suffered from post-herpetic neuralgia (commonly known as shingles). One of those patients enjoyed an essentially complete remission, with outstanding results. Certain other patients received transitory and partial benefits.

Thus, there has been shown and described a new and useful means for using sustained infusion of an NMDA receptor blocker drug, such as ketamine, for treating chronic intractable pain. Although this invention has been exemplified for purposes of illustration and description by reference to certain specific embodiments, it will be apparent to those skilled in the art that various modifications, alterations, and equivalents of the illustrated examples are possible. Any such changes which derive directly from the teachings herein, and which do not depart from the spirit and scope of the invention, are deemed to be covered by this invention.

### References

Carter, C., et al, “Ifenprodil and S L 82.0715 as cerebral anti-ischemic agents: II. Evidence for NMDA receptor antagonist properties,” J Pharmacol Exptl Ther 247: 1222-1232 (1988)
Correll, G. E., et al, “Subanesthetic ketamine infusion therapy: A retrospective analysis of a novel therapeutic approach to complex regional pain syndrome,” Pain Medicine 5: 263-275 (2004)
Deecher, D. C., et al, “Mechanisms of action of ibogaine and harmaline congeners based on radioligand studies,” Brain Res 571: 242-247 (1992)
Doze, V. A., et al, “Dexmedetomidine produces a hypnotic-anesthetic action in rats via activation of central alpha-2 adrenoceptors,” Anesthesiology 71: 75-79 (1989)
Eide, P. K., et al, “Relief of post-herpetic neuralgia with the NMDA receptor antagonist ketamine: a double-blind, cross-over comparison with morphine and placebo,” Pain 58: 347-54 (1994)
Eide, K. et al, “Continuous subcutaneous administration of the NMDA receptor antagonist ketamine in the treatment of post-herpetic neuralgia,” Pain 61: 221-8 (1995)
Fitzgibbon, E. J., et al, “Low dose ketamine as an analgesic adjuvant in difficult pain syndromes: a strategy for conversion from parenteral to oral ketamine,” J Pain Symptom Management 23: 165-70 (2002)
Goodman and Gilman, eds., The Pharmacological Basis of Therapeutics, eighth edition (Pergamon Press, 1990)
Grimwood, S., et al, “Characterisation of NMDA receptor-specific ifenprodil binding to recombinant human NR1a/NR2B receptors,” J Neurochem 75: 2455-63 (2000)
Harbut, R. E., et al, “Successful treatment of a nine-year case of complex regional pain syndrome . . . ” Pain Medicine 3: 147-155 (2002)
Harden, R. N., et al, “CRPS-I and CRPS-II: A Strategic View,” in Complex Regional Pain Syndrome: Progress in Pain Research and Management, volume 22 (IASP Press, Seattle, Wash., 2001)
Inaba, T., et al, “In vitro inhibition studies of two isozymes of human liver cytochrome P-450,” Drug Metabolism and Disposition 13: 443-447 (1985)
Janes, K. A., et al, “Polysaccharide colloidal particles as delivery systems for macromolecules,” Adv Drug Deliv Rev 23: 83-97 (2001)
Kannan, T. R., et al, “Oral ketamine as an adjuvant to oral morphine for neuropathic pain in cancer patients,” J Pain Symptom Management 23: 60-5 (2002)
Klepstad, P., et al, “Long-term treatment with ketamine in a 12-year-old girl with severe neuropathic pain caused by a cervical spinal tumor,” J Pediatr Hematol Oncol 23: 616-9 (2001)
Merskey, H., et al, Classification of Chronic Pain: Description of Chronic Pain Syndromes and Definitions of Pain Terms (IASP Press, Seattle, Wash., 1994)
Mitchell, A. C., “An unusual case of chronic neuropathic pain responds to an optimum frequency of intravenous ketamine infusions,” J Pain Symptom Manage 21: 443-6 (2001)
Rabben, T. et al, “Interindividual differences in the analgesic response to ketamine in chronic orofacial pain,” Eur J Pain 5: 233-40 (2001)
Ruffolo, R. R., et al, “Pharmacologic and therapeutic applications of α2-adrenoceptor subtypes,” Annu Rev Pharmacol Toxicol 32: 243-279 (1993)
Sanger, D. J., et al, “The anti-ischemic drug eliprodil blocks hyperactivity induced by intra-accumbens injection of spermine,” Behav Pharmacol 6: 520-526 (1995)
Schipper, N. G., et al, “Chitosans as absorption enhancers of poorly absorbable drugs,” Eur J Pharm Sci 8: 335-43 (1999)
Schmidt, C. J. et al, “Minireview: The role of 5HT-2A receptors in antipsychotic activity,” Life Sciences 56: 2209-2222 (1995)
Schwartzman, R. J., et al, “Reflex sympathetic dystrophy,” Curr Rheumatol Rep 4: 165-9 (2002)
Sheardown, M. J., et al, “Blockade of AMPA receptors in the CA1 region of the hippocampus prevents ischaemia induced cell death,” pp. 245-253 in Krieglstein, J., and Oberpichler, H., eds., Pharmacology of Cerebral Ischemia 1990 (Wissenschaftliche Verlagsgesellschaft, Stuttgart, Germany, 1990)
Tarnawa, I., et al, “GYKI 52466, an inhibitor of spinal reflexes, is a potent quisqualate antagonist,” pp. 538-546 in Lubec and Rosenthal (eds.), Amino Acids: Chemistry, Biology, and Medicine (ESCOM Science Publishers, Leiden, Netherlands, 1990)
Trapani, G., et al, “Propofol analogues: Synthesis, relationships between structure and affinity at GABA-A receptor in rat brain, and differential electrophysiological profile at recombinant human GABA-A receptors,” J Med Chemistry 41: 1846-54 (1998)

1. A method for treating chronic pain, comprising the steps of administering to a patient in need of such treatment:
a. at least one NMDA receptor antagonist drug, at a sustained dosage which is able to substantially reduce the patient's chronic pain, and over a continuous period of time, wherein that dosage level over that period of time has been shown in previous human treatments to provide long-lasting relief from chronic pain even after administration of the NMDA receptor antagonist drug has been discontinued; and,
b. at least one safener drug, at a concentration which has been shown, using in vivo animal tests, to reduce neurotoxic damage caused by potent NMDA receptor antagonist drugs in the absence of a safener drug.

2. The method of claim 1, wherein the NMDA receptor antagonist drug comprises ketamine, and wherein the ketamine is administered to the patient by intravenous infusion over a continuous period of at least 36 hours, at a dosage which is sufficient to establish and sustain a blood concentration of at least about 200 nanograms of ketamine per milliliter of blood plasma for a period of at least about 24 hours.

3. The method of claim 2, wherein the ketamine is administered to the patient at a dosage which is sufficient to establish and sustain a blood concentration of at least about 250 nanograms of ketamine per milliliter of blood plasma for a period of at least about 40 hours.

4. The method of claim 1, wherein the safener drug has also been shown in human treatments to potentiate the pain-relieving efficacy of the NMDA receptor antagonist drug.

5. The method of claim 4, wherein the safener drug comprises an alpha-2 adrenergic agonist drug.

6. The method of claim 5, wherein the alpha-2 adrenergic agonist drug is selected from the group consisting of clonidine, iodoclonidine, guanabenz, xylazine, medetomidine, tizanidine, rilmenidine, alpha-methyldopa, alpha-methylnoradrenaline, guanfacine, dexmedetomidine, azepexole, and lofexidine.

7. The method of claim 2, wherein the safener drug comprises an alpha-2 adrenergic agonist drug.

8. The method of claim 7, wherein the alpha-2 adrenergic agonist drug is selected from the group consisting of clonidine, iodoclonidine, guanabenz, xylazine, medetomidine, tizanidine, rilmenidine, alpha-methyldopa, alpha-methylnoradrenaline, guanfacine, dexmedetomidine, azepexole, and lofexidine.

9. The method of claim 1, wherein the safener drug acts as a direct agonist at GABA receptors even in the absence of naturally-occurring GABA.

10. The method of claim 9, wherein the direct GABA agonist is selected from the group consisting of propofol, pentobarbital, secobarbital, and thiamylal, and analogs and derivatives thereof which are active as direct GABA agonists.

11. The method of claim 1, wherein a water-soluble magnesium salt is also administered to the patient.

12. The method of claim 2, wherein a water-soluble magnesium salt is also administered to the patient.

13. A method for treating chronic pain, comprising the step of intravenously injecting into a patient in need of such treatment at least one NMDA receptor antagonist drug, at a sustained dosage over a period of time wherein that dosage over that period of time will provide lasting relief from chronic or neuropathic pain even after administration of the NMDA receptor antagonist drug has been discontinued.

14. The method of claim 13 wherein the NMDA receptor antagonist drug has an inherent safening activity due to activity at a second type of neuronal receptor.

15. The method of claim 14 wherein the NMDA receptor drug which has inherent safening activity is selected from the group consisting of ibogaine, ibogamine, eliprodil, ifenprodil, and analogs and derivatives thereof which are active as NMDA antagonists.

16. The method of claim 13 wherein the NMDA receptor antagonist drug comprises ketamine, and wherein the sustained dosage over a continuous period of time is able to establish ketamine levels in circulating blood plasma of at least about 200 nanograms of ketamine per milliliter of blood plasma, for at least 24 hours continuously.

17. The method of claim 13 wherein at least one safener drug which can reduce neurotoxic side effects of potent NMDA receptor antagonist drugs in animal tests is coadministered to the patient along with the NMDA receptor antagonist drug.

18. The method of claim 17, wherein the safener drug comprises an alpha-2 adrenergic agonist drug.

19. The method of claim 18, wherein the alpha-2 adrenergic agonist drug is selected from the group consisting of clonidine, iodoclonidine, guanabenz, xylazine, medetomidine, tizanidine, rilmenidine, alpha-methyldopa, alpha-methylnoradrenaline, guanfacine, dexmedetomidine, azepexole, and lofexidine.

20. The method of claim 13, wherein a water-soluble magnesium salt is also administered to the patient.

21. A composition of matter, comprising an injectable aqueous mixture of an NMDA receptor antagonist drug and an alpha-2 agonist drug, wherein each drug is present in the aqueous mixture at a concentration which is suited for intravenous infusion into a patient over a prolonged span of time in a dosage regimen capable of providing permanent relief from a neuropathic pain disorder.

22. The composition of matter of claim 21, wherein the NMDA receptor antagonist drug comprises ketamine, and wherein the injectable aqueous mixture also contains at least one safener drug that can reduce neurotoxic side effects of potent NMDA receptor antagonist drugs in animal tests.

23. The composition of matter of claim 22, wherein the safener drug comprises an alpha-2 adrenergic agonist drug.

24. The composition of matter of claim 21, wherein the injectable aqueous mixture also contains a magnesium salt.

25. An article of manufacture, comprising an injectable aqueous formulation containing at least one NMDA antagonist drug, enclosed within a package that maintains sterility of the aqueous formulation and that contains printed information stating that the NMDA antagonist drug is intended for sustained intravenous infusion for several days continuously, at a dosage which must be titrated individually for a specific patient who receives such treatment.

26. The article of manufacture of claim 25, wherein the aqueous formulation also contains at least one safener drug that can reduce neurotoxic side effects of potent NMDA receptor antagonist drugs in animal tests.

27. The article of manufacture of claim 25, wherein the aqueous formulation also contains a magnesium salt.
